(19) United States
(12) Patent Application Publication
(10) Pub. No.: US 2015/0051589 A1
(43) Pub. Date: Feb. 19, 2015

(54) CAPSULE TYPE MEDICAL DEVICE AND MEDICAL SYSTEM

(71) Applicant: SONY CORPORATION, Tokyo (JP)

(72) Inventors: Yoichiro Sako, Tokyo (JP); Akira Tange, Tokyo (JP); Takatoshi Nakamura, Kanagawa (JP); Yuki Koga, Tokyo (JP); Katsumi Ando, Tokyo (JP)

(73) Assignee: Sony Corporation, Tokyo (JP)

(21) Appl. No.: 14/386,357

(22) PCT Filed: Feb. 1, 2013
(86) PCT No.: PCT/JP2013/052343

§ 371 Date: Sep. 19, 2014

(30) Foreign Application Priority Data
Mar. 27, 2012 (JP) 2012-071829

(51) Int. Cl. A61K 9/00

(52) U.S. Cl. CPC A61K 9/0097
USPC 604/891.1

(57) ABSTRACT

Provided is a capsule type medical device which is able to be suspended in a body cavity. The capsule type medical device performs continuous treatment while being suspended at a predetermined site.

### TECHNICAL FIELD

The present disclosure relates to a capsule type medical device and a medical system.

### BACKGROUND ART

In recent years, a capsule type medical device to be introduced into a body of a test object is known. Such a known medical device photographs sites in the body at random, extracts a sample or the like from the body, or discharge a medicine, for example.

In particular, Patent Literatures 1 and 2 below each disclose a capsule type medical device which observes the progress of a predetermined site in a body cavity, the device being suspended at a desired position (a desired affected part) among sites in the body.

### CITATION LIST

### Patent Literature

Patent Literature 1: JP 2007-014634A
Patent Literature 2: JP 2005-204806A

### SUMMARY OF INVENTION

### Technical Problem

However, the capsule type medical devices disclosed in Patent Literatures 1 and 2 observe the progress of the predetermined site, but do not perform any treatment to the predetermined site (the affected part).

Accordingly, the present disclosure propose a capsule type medical device which performs more effective treatment while being suspended at a predetermined site and performing continuous treatment, and a medical system.

### Solution to Problem

According to the present disclosure, there is proposed a capsule type medical device which can be suspended in a body cavity and performs continuous treatment while being suspended at a predetermined site.

According to the present disclosure, there is proposed a medical system including a control device, and a capsule type medical device configured to be able to be suspended in a body cavity. The capsule type medical device includes a reception unit configured to receive a control signal from the control device, and a treatment unit configured to perform continuous treatment while being suspended at a predetermined site in accordance with the control signal received by the reception unit.

### Advantageous Effects of Invention

As described above, according to the present disclosure, it becomes possible to perform more effective treatment while being suspended at a predetermined site and performing continuous treatment.

### BRIEF DESCRIPTION OF DRAWINGS

FIG. 1 is a view showing an overview of a medical system according to an embodiment of the present disclosure.

FIG. 2 is a block diagram showing a configuration of a control device according to a first embodiment.

FIG. 3 is an outline drawing of a capsule type medical device according to a first embodiment.

FIG. 4 is a transition diagram of an internal structure of a capsule type medical device according to a first embodiment.

FIG. 5 shows an example of a suspension means of a capsule type medical device according to a first embodiment.

FIG. 6 is a flowchart showing a medicine discharging operation according to a first embodiment.

FIG. 7 shows an example of a medicine sprayed site specification screen according to a first embodiment.

FIG. 8 shows a case where a specific site is registered by use of a medicine sprayed site specification screen according to a first embodiment.

FIG. 9 shows an example of an internal structure and a suspension means of a capsule type medical device according to a modification example 1.

FIG. 10 is a flowchart showing a medicine discharging operation according to a modification example 1.

FIG. 11 shows a capsule type medical device according to a modification example 2.

FIG. 12 shows a configuration of a capsule type medical device according to a second embodiment.

FIG. 13 shows another suspension means according to the present disclosure.

### DESCRIPTION OF EMBODIMENTS

Hereinafter, preferred embodiments of the present invention will be described in detail with reference to the appended drawings. Note that, in this specification and the drawings, elements that have substantially the same function and structure are denoted with the same reference signs, and repeated explanation is omitted.

The description will be made in the following order.

1. Overview of medical system according to embodiment of present disclosure

2. Embodiments
2-1. First embodiment
   2-1-1. Configuration of control device
   2-1-2. Structure of capsule type medical device
   2-1-3. Continuous medicine discharging operation
   2-1-4. Specification of medicine sprayed site
   2-1-5. Modification example 1
   2-1-6. Modification example 2
2-2. Second embodiment

3. Conclusion

### 1. OVERVIEW OF MEDICAL SYSTEM ACCORDING TO EMBODIMENT OF PRESENT DISCLOSURE

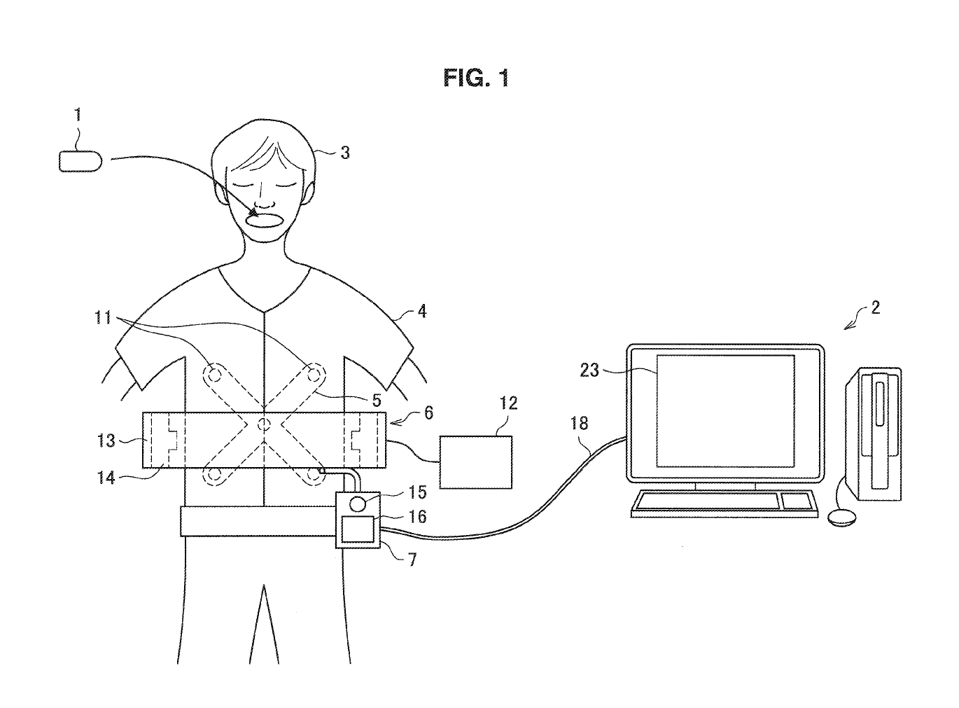

First, an overview of a medical system according to an embodiment of the present disclosure will be described with reference to FIG. 1. As shown in FIG. 1, the medical system according to an embodiment of the present disclosure includes a capsule type medical device 1 (hereinafter also referred to as capsule 1), a rotating magnetic field generating device 6, and a control device 2.

The capsule 1 is swallowed through the mouth of a test object 3, as shown in FIG. 1, and transmits an image signal (a photographed image) obtained when the capsule 1 photographs an inner wall of an intracelom pipeline optically when passing through the intracelom pipeline.

As shown in FIG. 1, further, the test object 3 wears a shield shirt 4. The shield shirt 4 has a shielding function, and includes an antenna unit 5 attached inside, the antenna unit 5 having a plurality of antennas 11. The antenna unit 5 outputs, to an extracorporeal unit 7 connected to the antenna unit 5, the received photographed image that is transmitted from the capsule 1 and received by the antennas 11.

The extracorporeal unit 7 is attached to a belt of the test object 3 with a detachable hook, for example, and retains the photographed image that is outputted from the antenna unit 5. Further, the extracorporeal unit 7 has a box shape as shown in FIG. 1, for example, and includes an operation button 15 for performing control operation and a liquid crystal monitor 16 for displaying an image, the operation button 15 and the liquid crystal monitor 16 being provided on a front surface of the extracorporeal unit 7.

The photographed image retained in the extracorporeal unit 7 may be displayed on the liquid crystal monitor 16 during or after testing, or may be transmitted to the control device 2 during or after testing so as to be displayed on a display unit 23 of the control device 2. The extracorporeal unit 7 and the control device 2 may be detachably connected to each other with a wire, for example, via a communication cable such as an USB cable 18, as shown in FIG. 1, or may be wirelessly connected to each other.

Accordingly, during or after testing, a medical staff can check the photographed image of the inside of the intracelom pipeline of the test object 3 with the liquid crystal monitor 16 of the extracorporeal unit 7 or the display unit 23 of the control device 2.

Further, as shown in FIG. 1, the rotating magnetic field generating device 6 is disposed on the periphery of the test object 3, such as a waist part of the test object 3. In the rotating magnetic field generating device 6, electromagnets 14 are arranged at a plurality of portions in the circumferential direction of a ring-shape frame member 13 and magnetic poles of the electromagnets 14 repel each other. The rotating magnetic field generating device 6 includes a driver circuit 12 which supplies driving signals to the electromagnets 14.

The capsule 1 according to the present embodiment has a structure such that a medicine is stored therein and allows the medicine to be discharged by generation of a rotating magnetic field. The rotating magnetic field is generated by the operation of the above described driver circuit 12 and sequential supply of direct current, as driving signals, from the driver circuit 12 to the electromagnets 14 at a plurality of portions.

Operation timing of the driver circuit 12 may be based on operation on a switch (not shown) of the driver circuit 12, the operation being made by a medical staff who has checked an affected part with the photographed image displayed on the liquid crystal monitor 16 of the extracorporeal unit 7 or the display unit 23 of the control device 2, for example. Alternatively, the driver circuit 12 may operate in accordance with an operation signal from the control device 2.

Here, the capsules disclosed in Patent Literatures 1 and 2 observe the progress of the predetermined site by being introduced into the body and suspended at the predetermined site, but do not perform any treatment to the predetermined site.

Accordingly, according to an embodiment of the present disclosure, there is provided a medical system that can perform more effective treatment by being suspended at a predetermined site and performing continuous treatment.

The overview of the medical system according to an embodiment of the present disclosure has been described above. Next, a plurality of embodiments of the medical system according to the present disclosure will be specifically described.

### 2. EMBODIMENTS

### 2-1. First Embodiment

As shown in FIG. 1, a medical system according to a first embodiment includes the capsule 1, which is to be introduced into the body of the test object 3, and the control device 2. A configuration of the control device 2, a structure of the capsule 1, continuous medicine discharging operation, and the like according to the first embodiment will be described in sequence below.

### 2-1-1. Configuration of Control Device

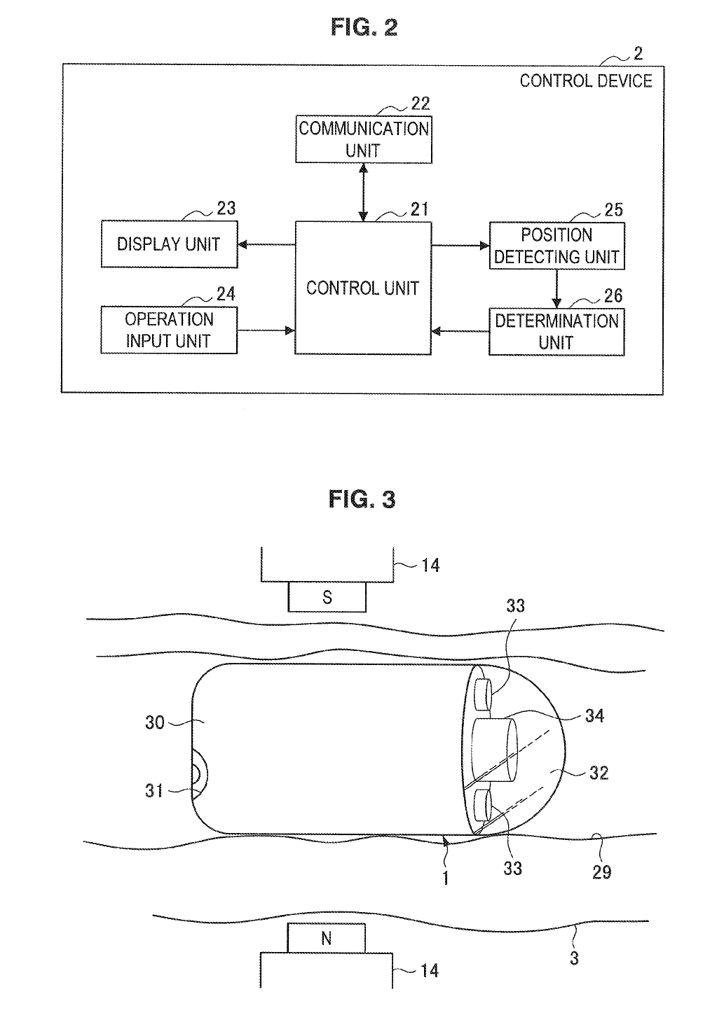

FIG. 2 is a block diagram showing the configuration of the control device 2 according to the first embodiment. As shown in FIG. 2, the control device 2 includes a control unit 21, a communication unit 22, the display unit 23, an operation input unit 24, a position detecting unit 25, and a determination unit 26.

The communication unit 22 is connected to an external device and has a function of transmitting and receiving data. For example, the communication unit 22 is connected to the extracorporeal unit 7 and receives the photographed image and signals for position detection from the capsule 1 via the extracorporeal unit 7. The communication unit 22 may notify the capsule 1 that the position of the capsule 1 has reached the specific site or the vicinity of the specific site. Further, the communication unit 22 may be connected to the rotating magnetic field generating device 6 with or without wires and may transmit an operation signal for operating the driver circuit 12 to the rotating magnetic field generating device 6 so that the capsule 1 can discharge the medicine.

The display unit 23 has a function of displaying a screen including images and texts under control of the control unit 21. Further, the display unit 23 is achieved by a liquid crystal display (LCD), an organic light-emitting diode (OLED), a cathode ray tube (CRT), or the like.

More specifically, the display unit 23 displays the photographed image received from the capsule 1 via the extracorporeal unit 7, for example. Thus, a medical staff can check the photographed image of the inside of the body of the test object 3 to recognize the position of the capsule 1 or determine whether or not the capsule 1 is located at a specific site where the medicine is to be discharged. Further, the display unit 23 may display a screen for specification for accepting registration of the specific site where a predetermined medicine is to be discharged. The specification of a medicine sprayed site by use of the screen for specification will be described later in detail in “2-2-4. Specification of medicine sprayed site.”

The operation input unit 24 has functions of detecting operation made by the medical staff and of outputting an input signal generated on the basis of the detected operation input to the control unit 21. The operation input unit 24 is achieved by a mouse, a keyboard, a touch panel, and the like. The medical staff can operate the operation input unit 24 to perform operation such as the registration of the specific site.

The control unit 21 has a function of controlling the whole control device 2. For example, the control unit 21 performs control such that the photographed image received by the communication unit 22 is displayed on the display unit 23.

The control unit 21 according to the present embodiment may control the communication unit 22 on the basis of determination results that are outputted from the determination unit 26 and may transmit an operation signal to the rotating magnetic field generating device 6. More specifically, in a case where the determination unit 26 determines that the capsule 1 has reached the medicine sprayed site or the vicinity of the medicine sprayed site which is specified in advance, the control unit 21 controls the communication unit 22 and transmits the operation signal to the rotating magnetic field generating device 6.

The position detecting unit 25 detects (calculates) the position of the capsule 1 on the basis of the signal for position detection, which is received by the communication unit 22 from the capsule 1. Here, the signal for position detection may be position information or the photographed image. The position detecting unit 25 may analyze the photographed image of the inside of the body, photographed by the capsule 1, to detect the position of the capsule 1. Further, the position detecting unit 25 outputs the detected position of the capsule 1 to the determination unit 26.

The determination unit 26 determines whether or not the position of the capsule 1, which is detected by the position detecting unit 25, is at the medicine sprayed site or in the vicinity of the medicine sprayed site which is specified in advance, and outputs the determination results to the control unit 21. Note that, as described above, the specific site where the medicine is to be sprayed in the body may be registered in advance by the medical staff.

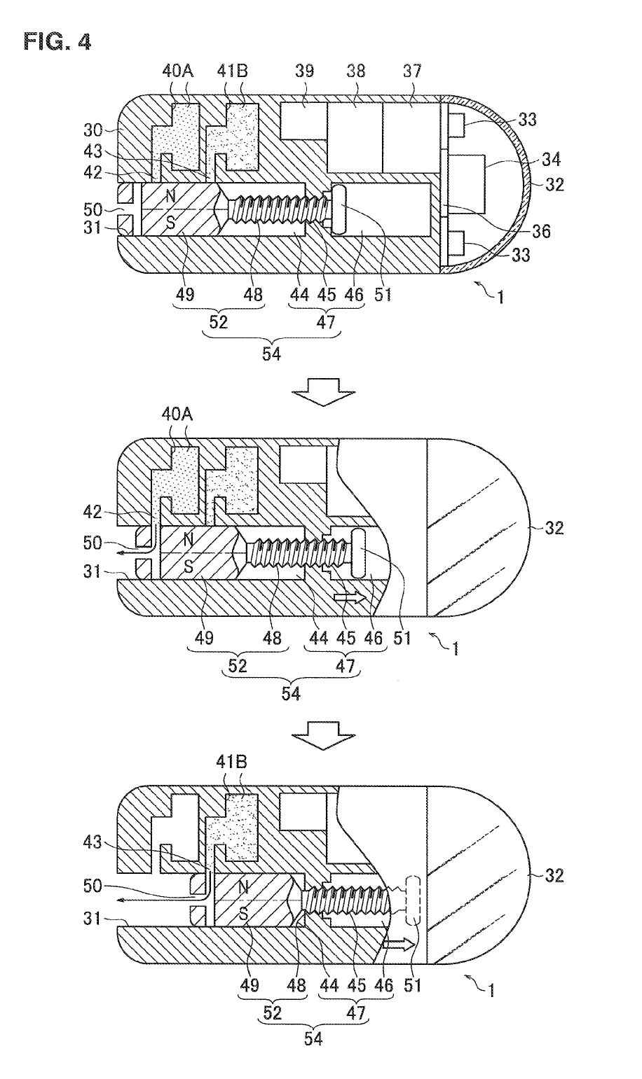

The configuration of the control device 2 according to the first embodiment has been described above in detail. Next, the structure of the capsule 1 according to the first embodiment will be described with reference to FIG. 3 and FIG. 4.

### 2-1-2. Structure of Capsule Type Medical Device

FIG. 3 is an outline drawing of the capsule 1 according to the first embodiment. As shown in FIG. 3, the capsule 1 to be inserted into an intracelom pipeline 29 of the test object 3 has a substantially cylindrical shape, and is covered with an outer case 30 which is choked by a curved rear end of the capsule 1. Further, a rear end portion of the outer case 30 is provided with an opening 31 through which the medicine is to be discharged, and a hemispherical transparent cover 32 is watertightly connected and secured to a tip portion of the outer case 30.

Inside a container that is sealed hermitically inside the transparent cover 32, as shown in FIG. 3, a photographing optical system 34 is disposed at the center facing the transparent cover 32, and lighting units such as white LEDs 33 are disposed on the periphery of the photographing optical system 34.

In a case where the capsule 1 reaches the predetermined site or the vicinity of the predetermined site in the body, driving current is supplied to the plurality of electromagnets 14 of the rotating magnetic field generating device 6, which are arranged on the periphery of the site, so that a rotating magnetic field is generated and the medicine is discharged through the opening 31 of the capsule 1.

More specifically, a moving body 52 provided inside the capsule 1 to be rotatable is caused to move by the rotating magnetic field, so that a state in which a storage unit inside the capsule 1 is choked becomes a state in which the storage unit communicates with the outside, and thereby the medicine stored in the storage unit is discharged. Such an internal structure of the capsule 1 will be specifically described below with reference to FIG. 4.

FIG. 4 is a transition diagram of an internal structure from a state where a moving body inside the capsule 1 chokes a plurality of storage units (initial state) to a state where the respective plurality of storage units are made to communicate with the outside. First, with reference to the diagram of the initial state shown in the upper part of FIG. 4, the internal structure of the capsule 1 will be described.

As shown in the upper part of FIG. 4, at a position where an image is formed in the photographing optical system 34 disposed in the center portion facing the transparent cover 32, a photographing sensor 36 such as a CMOS imager (or a CCD) is disposed.

In a backward upper portion of the photographing sensor 36, a control unit 37, a memory and communication unit 38, and a battery 39 are disposed.

The control unit 37 drives the photographing sensor 36, performs signal processing of an output signal of the photographing sensor 36, and controls other circuits such as the memory and communication unit 38 which will be described next. Further, the control unit 37 according to the present embodiment performs control such that a suspension means (not shown) operates in a case where the capsule 1 has reached the specific site or the vicinity of the specific site. The operation of the suspension means can suspend the capsule 1 at the specific site or in the vicinity of the specific site.

The memory and communication unit 38 has a function of memorizing the photographed image signal (the photographed image) and a communication function of transmitting the image signal wirelessly, for example.

The battery 39 has a button shape, for example, is in conduction with a wiring substrate that is not shown, and is electrically connected to the memory and communication unit 38 via the wiring substrate.

As shown in FIG. 4, storage units 40 and 41 are provided in a portion that is shielded from the battery 39, the memory and communication unit 38, and the control unit 37 by a wall portion due to the outer case 30, the portion being located at the backward (left) of the battery 39.

The respective medicines to be stored in the storage units 40 and 41 are inserted in advance, in addition to a pressurized gas, through a horizontal hole that is not shown. The horizontal hole is choked with a rubber stopper or the like after the medicine is inserted.

As shown in FIG. 4, the storage units 40 and 41 are provided eccentrically in the upward direction from the center axis of the capsule 1. Meanwhile, a medicine discharging unit 54, which discharges the medicine selectively from respective the storage units 40 and 41, is provided eccentrically in the downward direction from the center axis of the capsule 1 on the side opposite to the storage units 40 and 41.

The medicine discharging unit 54 according to the first embodiment, as shown in FIG. 4, is achieved by the moving body 52 and a moving body storing unit 47 which disposes or supports the moving body 52 such that the moving body 52 can rotatably move in the longitudinal direction of the capsule 1.

The moving body storing unit 47 includes a first depressed portion 44 having the opening 31 as a rear end, a screw hole (a female screw) 45 formed on a front end side of the first depressed portion 44, and a second depressed portion 46 which communicates with the first depressed portion 44 through the screw hole 45. Note that on a side portion of the first depressed portion 44, an opening 42 and an opening 43 of pipelines that communicate with the storage unit 40 and the storage unit 41, respectively, are open.

The moving body storing unit 47 stores and supports the moving body 52 in a state where a screw portion (a male screw portion) 48 provided on the front end side of the moving body 52 is screwed into the screw hole 45.

The moving body 52 is provided with the screw portion 48 on the front end side, as described above, and at the tip of the screw portion 48 (the front end of the moving body 52), for example, a disk-shape stopper 51 is further provided. The stopper 51 is stored inside the second depressed portion 46. Further, on the rear end side of the moving body 52, a cylindrical portion 49 which fits the first depressed portion 44 is provided, and in the vicinity of the rear end of the cylindrical portion 49, for example, a T-shape hole 50 is provided.

In the initial state shown in the upper part of FIG. 4, the moving body 52 chokes the openings 42 and 43 with the cylindrical portion 49. Here, the moving body 52 is formed of a permanent magnet in which both sides of the center axis shown by a dashed line (e.g., the upper part and the lower part of the center axis) are magnetized with N and S, for example. Accordingly, in the state shown in the upper part of FIG. 4, the generation of the rotating magnetic field by the electromagnets 14 shown in FIG. 3 causes the moving body 52 to rotate and to move to a tip direction (the right direction), as shown in the middle part of FIG. 4.

In the state shown in the middle part of FIG. 4, the T-shape hole 50 of the moving body 52 communicates with the opening 42, and a first medicine (hereinafter referred to as medicine A) stored in the storage unit 40 is discharged to the outside of the capsule 1 through the opening 42 and the T-shape hole 50.

Further, the moving body 52 rotates, and moves to the tip direction (the right direction) to the position where the stopper 51 touches a wall surface of the second depressed portion 46, for example. Then, as shown in the lower part of FIG. 4, the T-shape hole 50 of the moving body 52 communicates with the opening 42. Thus, a second medicine (hereinafter referred to as medicine B) stored in the storage unit 41 is discharged to the outside of the capsule 1 through the opening 43 and the T-shape hole 50.

As described above with reference to FIG. 4, in the present embodiment, the moving body 52 included in the medicine discharging unit 54 inside the capsule 1 rotates and moves in accordance with the generation of the external rotating magnetic field, so that the openings 42 and 43 which communicate with the storage units 40 and 41 storing the medicines are open and closed. Thus, the capsule 1 can discharge the medicines A and B sequentially to the outside.

Here, the capsule 1 according to the present embodiment can cause the control unit 37 to operate the suspension means so as to be suspended at the specific site or in the vicinity of the specific site, as described above. The suspension means can be achieved by various methods; for example, a balloon type may be used as described in JP 2003-325438A, for example. In a case of a balloon type, an elastic and airtight balloon covers a circumferential surface at the center of the capsule 1 in the longitudinal direction, and both ends of the balloon is airtightly secured to the circumferential surface of the capsule 1 with a belt-shape fixing member. In a case where the capsule 1 has reached the specific site or the vicinity of the specific site, in accordance with the control by the control unit 37 or the external rotating magnetic field, a pressurized gas stored in the capsule 1 flows into the balloon side, and the balloon expands. Thus, the capsule 1 can be suspended at the specific site or in the vicinity of the specific site.

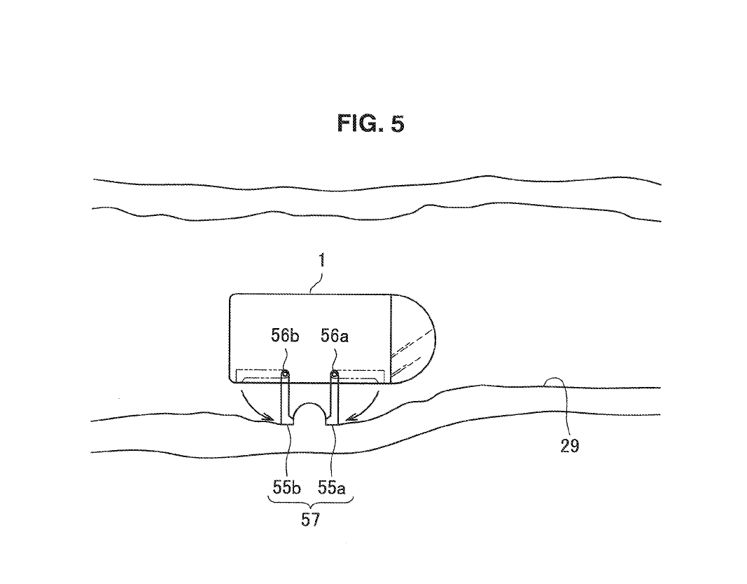

Further, it is possible to use an arm type as described in Patent Literature 2 (JP 2005-204806A) above. A specific description will be made below with reference to FIG. 5.

FIG. 5 shows an example of a suspension means of the capsule 1 according to the present embodiment. As shown in FIG. 5, the capsule 1 includes a suspension means 57 including arms 55a and 55b and pins 56a and 56b. The arms 55a and 55b provided at the center of the longitudinal direction of the capsule 1 rotates to be substantially vertical to the longitudinal direction of the capsule 1, from the state of being retained inside the capsule 1, due to an added force of springs (not shown) provided for the pins 56a and 56b.

In this manner, the arms 55a and 55b rotate with a fulcrum of the pins 56a and 56b, and pinch a mucous membrane of the inner wall of the intracelom pipeline 29, as shown in FIG. 5, so that the capsule 1 can be suspended at the specific site or in the vicinity of the specific site. Then, while being suspended at the specific site or in the vicinity of the specific site, the capsule 1 can perform continuous treatment.

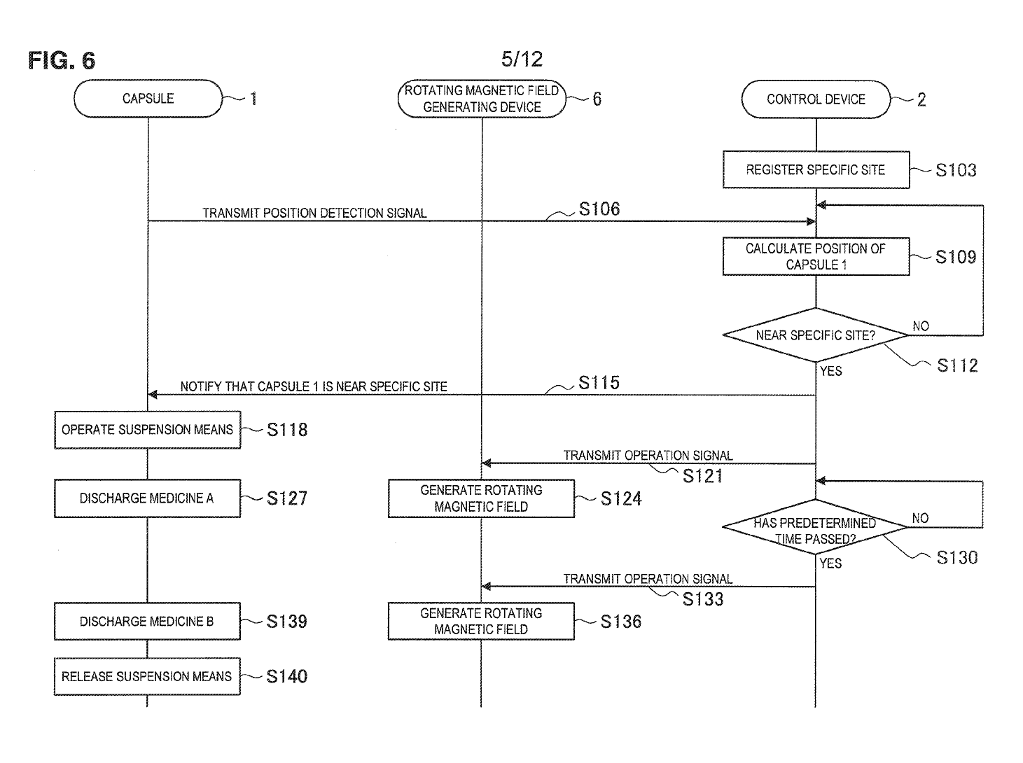

The structure of the capsule 1 according to the present embodiment has been described above in detail. Next, operation of the capsule 1 having the above structure will be specifically described with reference to FIG. 6. Note that in the present embodiment, as an example of continuous treatment, treatment in which a medicine is continuously discharged is performed.

### 2-1-3. Continuous Medicine Discharging Operation

FIG. 6 is a flowchart showing a medicine discharging operation according to the first embodiment. As shown in FIG. 6, first, in step S103, the control device 2 registers the specific site where the medicine is to be discharged (sprayed or applied). More specifically, the control device 2 memorizes the specific site where the medicine is to be discharged in association with the medicine to be discharged at the specific site on the basis of an operation input made by the medical staff, for example.

Next, in step S106, power is applied to the capsule 1, and the capsule 1 swallowed by the test object 3 transmits a position detection signal to the control device 2 while moving in the body cavity.

Next, in step S109, the position detecting unit 25 of the control device 2 detects the position of the capsule 1 on the basis of the intensity of the position detection signal transmitted from the capsule 1. Note that in a case where the photographed image is transmitted as the position detection signal from the capsule 1, the position detecting unit 25 may detect the position of the capsule 1 by analyzing the photographed image.

Next, in step S112, the determination unit 26 of the control device 2 determines whether or not the position of the capsule 1 detected by the position detecting unit 25 is the specific site or the vicinity of the specific site which is registered in the step S103. For example, the determination unit 26 determines that the capsule 1 has reached the specific site or the vicinity of specific site in a case where the capsule 1 is within a predetermined distance from the specific site registered in advance.

Next, in a case where the determination unit 26 determines that the capsule 1 has reached the specific site or the vicinity of the specific site, in step S115, the control device 2 notifies the capsule 1 that the capsule 1 is at the specific site or in the vicinity of the specific site.

Next, in step S118, the capsule 1 causes the suspension means to operate in response to the notification received from the control device 2. Specifically, as described above, for example, the capsule 1 may rotate the arms 55a and 55b and pinch the mucous membrane of the inner wall in the body cavity, so as to be suspended.

Further, in step S121, the control device 2 performs control such that an operation signal is transmitted to the rotating magnetic field generating device 6 so that the capsule 1 can discharge the medicine A.

Next, in step S124, the rotating magnetic field generating device 6 causes the driver circuit 12 to operate in accordance with the operation signal, and sequentially supplies direct current as driving signals to the electromagnets 14 at a plurality of portions from the driver circuit 12, so that the rotating magnetic field is generated.

Next, in step S127, the capsule 1 discharges the medicine A. As described above, when the rotating magnetic field is applied, the moving body 52 of the capsule 1 rotates and moves, and as shown in the middle part of FIG. 4, the opening 42 of the storage unit 40 storing the medicine A communicates with the outside through the T-shape hole 50 of the moving body 52. Accordingly, the medicine A of the storage unit 40 is discharged to the outside of the capsule 1 and the medicine A is discharged to the specific site and the vicinity of the specific site.

Next, in step S130, the control device 2 determines whether or not a predetermined time has passed after transmitting, to the rotating magnetic field generating device 6, the operation signal for causing the medicine A to be discharged.

After the predetermined time passes, in step S133, the control device 2 performs control such that an operation signal is transmitted to the rotating magnetic field generating device 6 so that the capsule 1 can discharge the medicine B.

Next, in step S136, the rotating magnetic field generating device 6 operates the driver circuit 12 in accordance with the operation signal, and sequentially supplies direct current from the driver circuit 12 to the electromagnets 14 at a plurality of positions, so as to generate a rotating magnetic field.

Next, in step S139, the capsule 1 discharges the medicine B. As described above, when the rotating magnetic field is applied, the moving body 52 inside the capsule 1 further rotates and moves, and as shown in the lower part of FIG. 4, the opening 43 of the storage unit 41 storing the medicine B communicates with the outside through the T-shape hole 50 of the moving body 52. Accordingly, the medicine B of the storage unit 41 is discharged to the outside of the capsule 1 and the medicine B is discharged to the specific site and the vicinity of the specific site.

Then, in step S140, the capsule 1 releases the suspension means, and starts to move in the body cavity to be exhausted to the outside.

As described above, by continuous medicine discharging operation of the capsule 1 according to the first embodiment, the plurality of medicines A and B can be discharged to the specific site or the vicinity of the specific site periodically at predetermined time intervals. Note that the timing of discharging plurality of medicines according to the present disclosure is not limited to periodical timing, and may be non-periodical timing. For example, the capsule according to the present embodiment may discharge a medicine for plural times of non-periodical timing, such as “six hours, six hours, and twelve hours” in a day. In addition, the capsule according to the present embodiment may discharge a medicine for plural time of non-periodical timing, such as “five minutes, five minutes, seven hours and fifty minutes, five minutes, five minutes, seven hours and fifty minutes, five minutes, five minutes, seven hours and fifty minutes, five minutes, and five minutes” (three times of medicine discharge for every five minutes at 8-hour intervals) in a day.

### 2-1-4. Specification of Medicine Sprayed Site

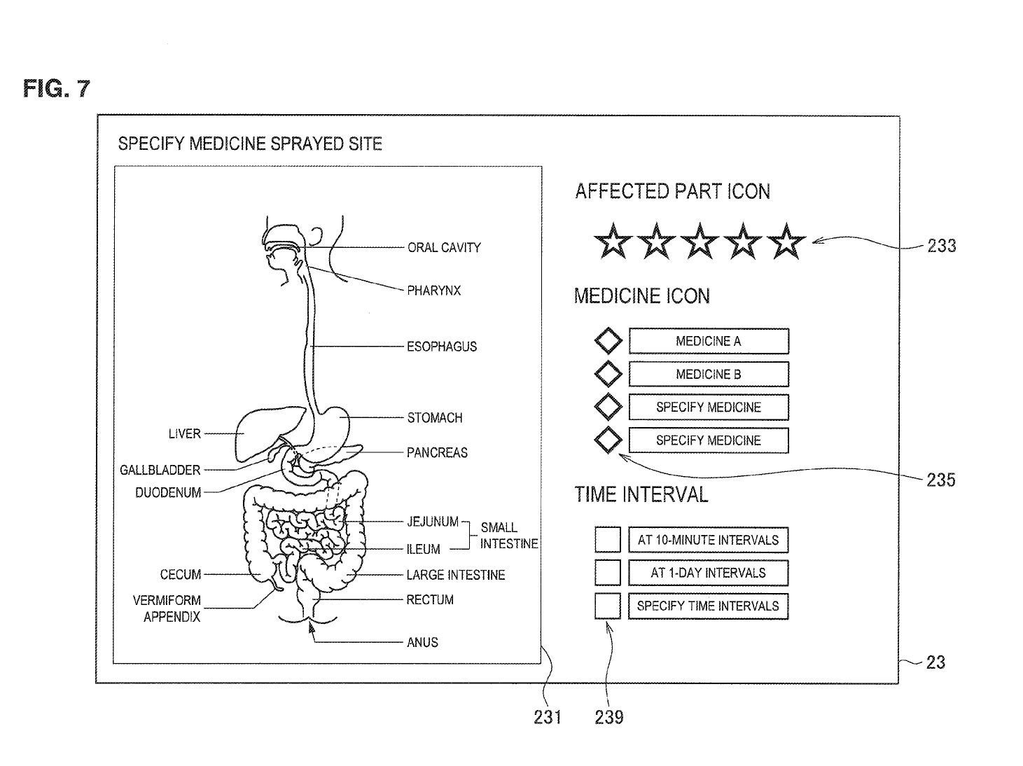

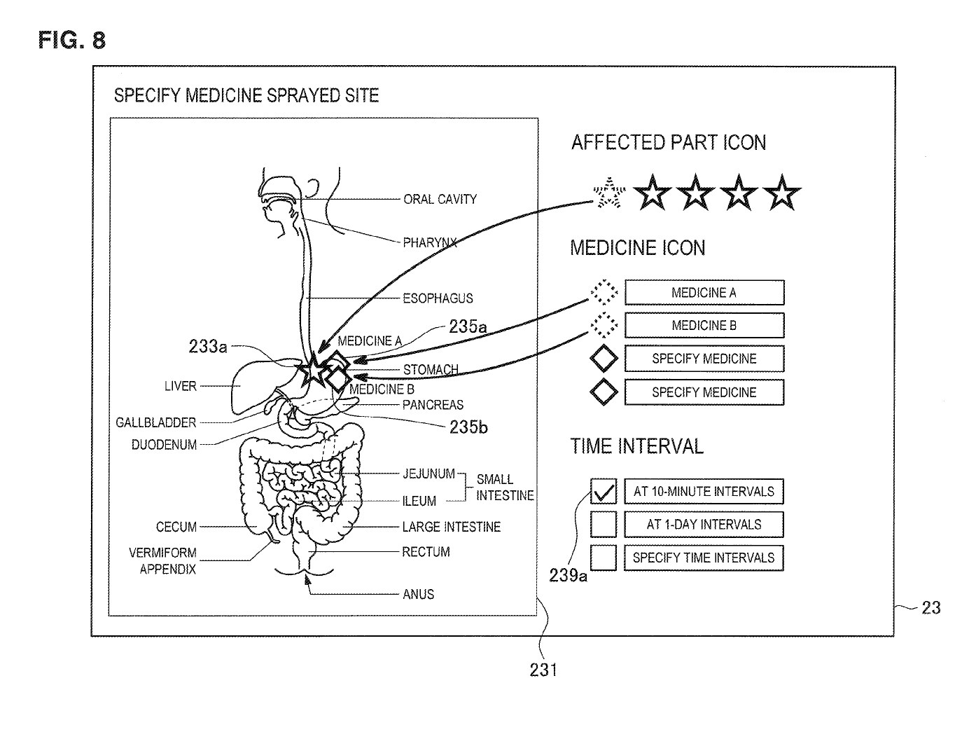

Next, registration of the specific site shown in the step S103 in FIG. 6 will be described. The medical staff can register intuitively the specific site where each medicine is to be sprayed in accordance with a specification screen displayed on the display unit 23 of the control device 2. An example of the specification screen will be specifically described below with reference FIG. 7 and FIG. 8.

FIG. 7 shows an example of a medicine sprayed site specification screen which is displayed on the display unit 23 of the control device 2 according to the first embodiment. As shown in FIG. 7, the medicine sprayed site specification screen includes a site image 231 representing sites in the body, an affected part icon 233, a medicine icon 235, and a checkbox 239 for selecting time intervals.

The site image 231 may be an image in which an illustration of each site is in association with the name thereof, as shown in FIG. 7. Note that the example shown in FIG. 7 shows a standard illustration of sites of the body as the site image 231; however, an actual position of the body of the test object 3 is substantially fixed, and the absolute position (the position coordinate) of each internal organ (each site) has been recognized already by the control unit 21 of the control device 2. Accordingly, the control unit 21 can calculate the position coordinate of the site of the test object 3, which corresponds to each site represented in the site image 231.

The affected part icon 233 is an icon for specifying the site to which the medicine is to be sprayed. Further, the medicine icon 235 is an icon for specifying the medicine that is to be sprayed to the specific site or the vicinity of the specific site.

The checkbox 239 for selecting time intervals is a checkbox for selecting time intervals between medicine discharges from the capsule to be introduced into the test object 3. In the example shown in FIG. 7, it is possible to select between “at 10-minute intervals” or “at 1-day intervals” or to input a given time interval by selecting “specify time intervals”.

Next, the registration of the specific site, the specification of the medicine that is to be sprayed to the specific site, and the selection of time intervals will be described with reference to FIG. 8. As shown in FIG. 8, the medical staff selects an affected part icon 233a and moves the affected part icon 233a to a desired specific site by performing a drag and drop operation.

The medical staff also specifies the medicine to be discharged at the specific site or in the vicinity of the specific site. Here, for example, the medical staff selects medicine icons 235a and 235b which represent the medicines A and B to be sprayed to the specific site or the vicinity of the specific site, and moves the selected capsule icons 235a and 235b to the affected part icon 233a or the vicinity of the affected part icon 233a by performing a drag and drop operation.

Further, the medical staff selects the period (time intervals) of medicine discharge operation. For example, in a case of causing a plurality of capsules to discharge the medicines A and B at 10-minute intervals, as shown in FIG. 8, a medical staff checks the checkbox 239a.

On the basis of the input operation made by the medical staff, the control unit 21 of the control device 2 calculates the actual coordinate position in the body of the test object 3, which corresponds to the specific site on the site image 231 on which the affected part icon 233a is moved. Further, the control unit 21 memorizes the type of medicines represented as the medicine icons 235a and 235b which are moved to the specific site or the vicinity of the specific site on the site image 231 and the type of the capsule storing these medicines in association with the calculated coordinate position of the specific site.

In this manner, the medical staff can specify the specific site intuitively by selecting the affected part icon 233 and the medicine icon 235 on the specification screen displayed on the display unit 23 of the control device 2, and by moving the affected part icon 233 and the medicine icon 235 to given positions on the site image 231.

As described above, in the first embodiment of the present disclosure, the control device 2 transmits an operation signal that allows the rotating magnetic field generating device 6 to generate the rotating magnetic field for every predetermined times, and accordingly, the capsule 1 can discharge the medicines A and B to the specific site or the vicinity of the specific site for every predetermined times (periodically or non-periodically).

### 2-1-5. Modification Example 1

Although, as the structure of the capsule 1 according to the first embodiment described above, the structure in which the moving body 52 rotates and moves in accordance with the external rotating magnetic field has been described with reference to FIG. 3 and FIG. 4, the structure of the capsule type medical device according to the present embodiment is not limited thereto.

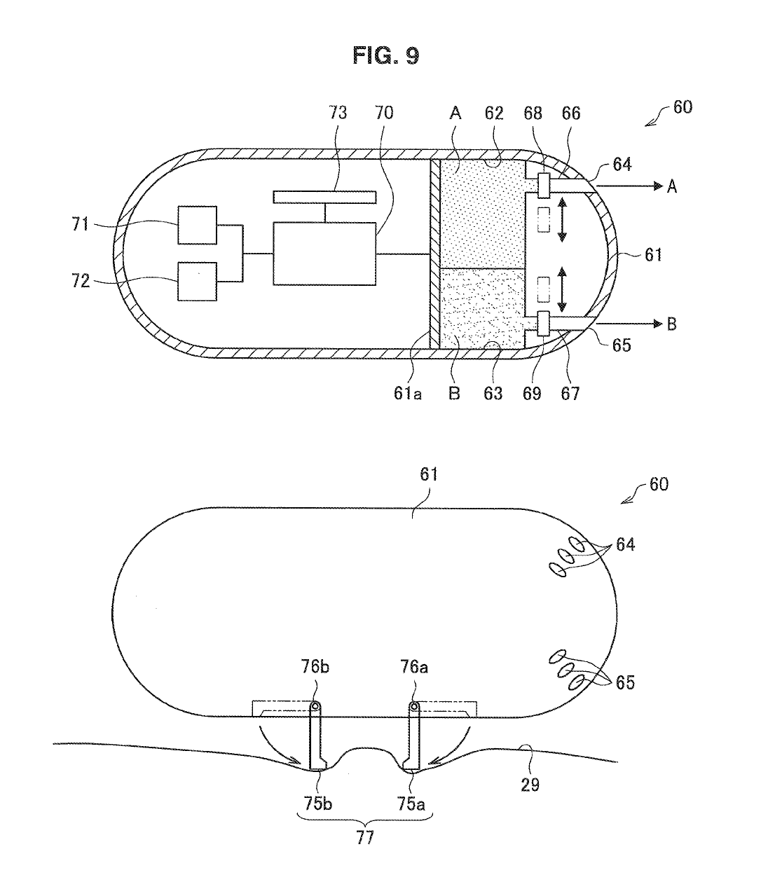

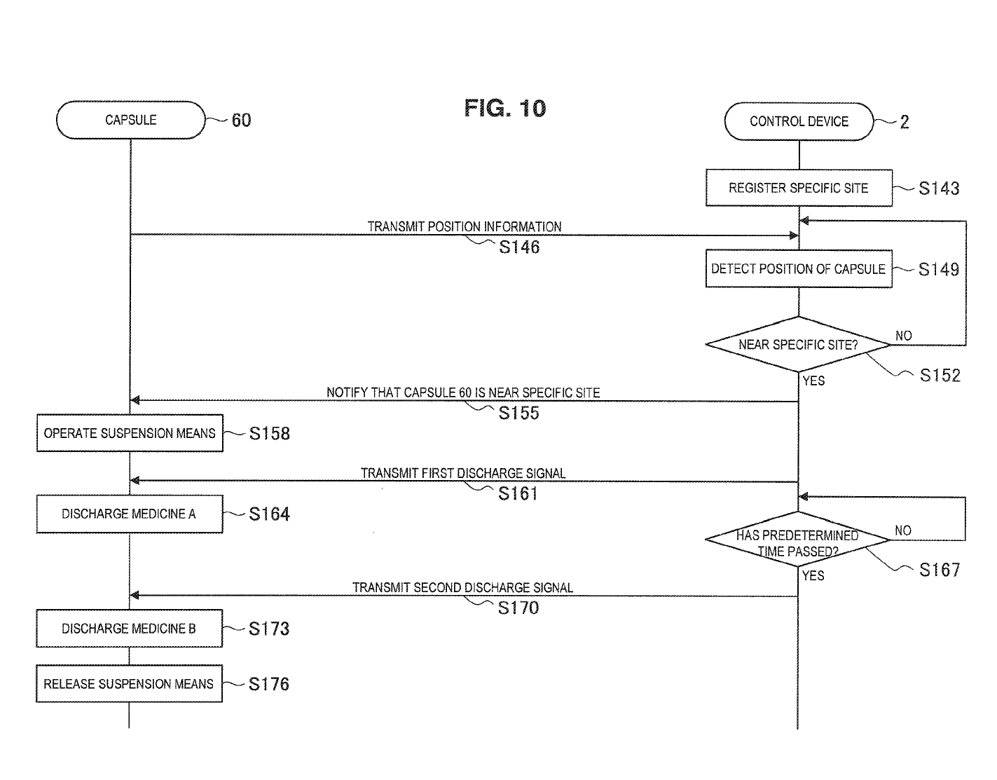

For example, the capsule type medical device according to the present disclosure may have a structure including a valve that opens and closes a discharge outlet of the medicine, the structure allowing the medicine to be discharged by controlling the valve. As a modification example of the first embodiment, a capsule type medical device including the valve that opens and closes the discharge outlet of the medicine will be specifically described below with reference to FIG. 9 and FIG. 10. Note that a medical system according to this modification example includes a capsule type medical device 60 (hereinafter referred to as capsule 60) storing the medicines A and B and the control device 2.

### —Structure

FIG. 9 shows an internal structure of a main unit of the capsule 60 according to the modification example 1 and an example of a suspension means of the capsule 60. As shown in the upper part of FIG. 9, the capsule 60 according to the present embodiment includes a battery 73, a control unit 70, a reception unit 71, and a transmission unit 72 inside a capsular housing 61 and on the left side of a wall portion 61a. Note that the housing 61 is formed with plastic or the like so as to seal the inside thereof hermitically.

Further, on the right side of the wall portion 61a, storage units 62 and 63 each storing the medicine, medicine discharging outlets 64 and 65 formed on the outer surface of the housing 61, medicine discharging pipelines 66 and 67 which communicate with the respective storage units and the respective medicine discharging outlets, and switch valves 68 and 69 which open and close the respective pipelines 66 and 67 are provided. Note that a plurality of the medicine discharging outlets 64 and 65 may be formed around the axis of the housing 61 on one end side.

Each structural element of the capsule 60 shown in the upper part of FIG. 9 will be described below. The battery 73 is a button-type battery, for example, and supplies power to each of the following structural elements: the control unit 70, the reception unit 71, and the transmission unit 72.

The control unit 70 has a function of controlling the whole capsule 60. The control unit 70 (a medicine discharging unit) according to the present embodiment causes the switch valve 68 or 69 to operate in accordance with a discharge signal (a control signal) received by the reception unit 71 and discharges the medicine A or medicine B. Further, the control unit 37 according to the present embodiment performs control such that the suspension means (refer to a suspension means 77 shown in the lower part of FIG. 9) to operate in a case where the capsule 60 has reached the specific site or the vicinity of the specific site. The operation of the suspension means allows the capsule 60 to be suspended at the specific site or the vicinity of the specific site.

The reception unit 71 has a function of receiving data from an external device. For example, the reception unit 71 receives the medicine discharge signal from the control device 2. Further, the reception unit 71 outputs the received medicine discharge signal to the control unit 70. The transmission unit 72 has a function of transmitting data to the external device. For example, the transmission unit 72 transmits a radio wave (a position detection signal) as position information indicating the position of the capsule 60 to the control device 2.

Note that in the example shown in the upper part of FIG. 9, the reception unit 71 and the transmission unit 72 are shown as separate blocks; however, the structure of the capsule 60 is not limited to the example shown in the upper part of FIG. 9, and may be a structure including a communication unit having a reception function and a transmission function, for example.

The storage unit 62 stores the medicine A. As shown in the upper part of FIG. 9, the storage unit 62 is connected to the medicine discharging pipeline 66. As described above, the switch valve 68 which is movable so as to open and close the medicine discharging pipeline 66 is provided.

Meanwhile, the storage unit 63 stores the medicine B. As shown in the upper part of FIG. 9, the storage unit 93 is connected to the medicine discharging pipeline 67. As described above, the switch valve 69 which is movable so as to open and close the medicine discharging pipeline 67 is provided.

The capsule 60 having such a structure is set at such a position that both the switch valves 68 and 69 choke both the pipelines 66 and 67 at an initial state. Then, in accordance with the discharge signal (the control signal) received by the reception unit 71 from the control device 2, the control unit 70 controls the switch valves 68 and 69 such that the pipelines 66 and 67 are open, and then each medicine is discharged.

For example, in a case where the reception unit 71 has received a first discharge signal, the control unit 70 can control the switch valve 68 such that the medicine discharging pipeline 66 is open, and can cause the medicine A to be discharged. Further, in a case where the reception unit 71 has received a second discharge signal, the control unit 70 can control the switch valve 69 such that the medicine discharging pipeline 67 is open, and can cause the medicine B to be discharged.

As described above with reference to the upper part of FIG. 9, in the present embodiment, the control unit 70 of the capsule 60 controls the switch valves 68 and 69, thereby enabling the medicines A and B to be discharged to the outside.

Here, the capsule 60 according to this modification example also causes the control unit 70 to operate the suspension means so as to be suspended at the specific site or in the vicinity of the specific site, as described above. The suspension means can be achieved by a variety of methods, and may be a balloon type as disclosed in JP 2003-325438A or an arm type as disclosed in the above Patent Literature 2 (JP 2005-204806A), for example. A specific description will be made below with reference to the lower part of FIG. 9.

The lower part of FIG. 9 shows an example of the suspension means of the capsule 60 according to the modification example 1. As shown in the lower part of FIG. 9, a plurality of the medicine discharging outlets 64 and 65 are formed around the axis on one end side of the housing 61.

Further, as shown in the lower part of FIG. 9, the capsule 60 includes the suspension means 77 including arms 75a and 75b and pins 76a and 76b. The arms 75a and 75b provided at the center of the longitudinal direction of the capsule 60 rotates to be substantially vertical to the longitudinal direction of the capsule 60 from the state of being retained inside the capsule 60 due to an added force of springs (not shown) provided for the pins 76a and 76b.

In this manner, the arms 75a and 75b rotate with a fulcrum of the pins 76a and 76b, and pinch a mucous membrane of the inner wall of the intracelom pipeline 29 so that the capsule 60 can be suspended at the specific site or in the vicinity of the specific site. Then, in the state of being suspended at the specific site or in the vicinity of the specific site, the capsule 60 can perform continuous treatment.

The structure of the capsule 60 according to this modification example has been described above in detail. Next, operation of this modification example will be specifically described with reference to FIG. 10. Note that in this embodiment, as an example of continuous treatment, treatment in which a medicine is discharged continuously is performed.

### —Cooperative Operation

FIG. 10 is a flowchart showing medicine discharging operation according to the modification example 1. Since steps S143, S149, and S152 shown in FIG. 10 are the same as the steps S103, S109, and S112 shown in FIG. 6, a description thereof is omitted here.

Further, in step S146, power is applied to the capsule 60, and the capsule 60 swallowed by the test object 3 transmits position information to the control device 2 while moving in the body cavity.

Next, in a case where the determination unit 26 of the control device 2 determines that the capsule 1 has reached the specific site or the vicinity of the specific site, in step S155, the control device 2 notifies the capsule 1 that the capsule 1 is at the specific site or in the vicinity of the specific site.

Next, in step S158, the capsule 60 causes the suspension means to operate in response to the notification received from the control device 2. Specifically, as described above, for example, the capsule 60 may rotate the arms 75a and 75b and pinch the mucous membrane of the inner wall in the body cavity, so as to be suspended.

Further, in step S161, the control device 2 performs control such that a first discharge signal (a control signal) is transmitted to the capsule 60 so that the capsule 60b can discharge the medicine A.

Next, in step S164, the capsule 60 controls the switch valve 68 in accordance with the first discharge signal received from the control device 2, and discharges the medicine A stored in the storage unit 62 to the specific site or the vicinity of the specific site.

Next, in step S167, the control device 2 determines whether or not a predetermined time has passed after transmitting, to the capsule 60, the first discharge signal for causing the medicine A to be discharged.

After the predetermined time passes, in step S170, the control device 2 performs control such that a second discharge signal (a control signal) is transmitted to the capsule 60 so that the capsule 60 can discharge the medicine B.

Next, in step S173, the capsule 60 controls the switch valve 69 in accordance with the second discharge signal received from the control device 2, and discharges the medicine B stored in the storage unit 63 to the specific site or the vicinity of the specific site.

Then, in step S176, the capsule 60 releases the suspension means, and starts to move in the body cavity again to be exhausted to the outside.

As described above, in the continuous medicine discharging operation performed by the capsule 60 according to the modification example 1, at the specific site or the vicinity of the specific site, the plurality of medicines A and B can be discharged periodically or non-periodically at predetermined time intervals.

### 2-1-6. Modification Example 2

The above embodiment and the modification example 1 have shown periodical medicine discharge (at time intervals) as continuous medicine discharging operation; however, continuous medicine discharging operation according to the present disclosure is not limited thereto. For example, as continuous medicine discharging operation, a medicine discharging outlet may be made smaller and a small amount of medicine may be repeatedly discharged. Further, as continuous medicine discharging operation, the size of the medicine discharging outlet may be controlled so that a discharge range may be narrowed in accordance with the passage of time.

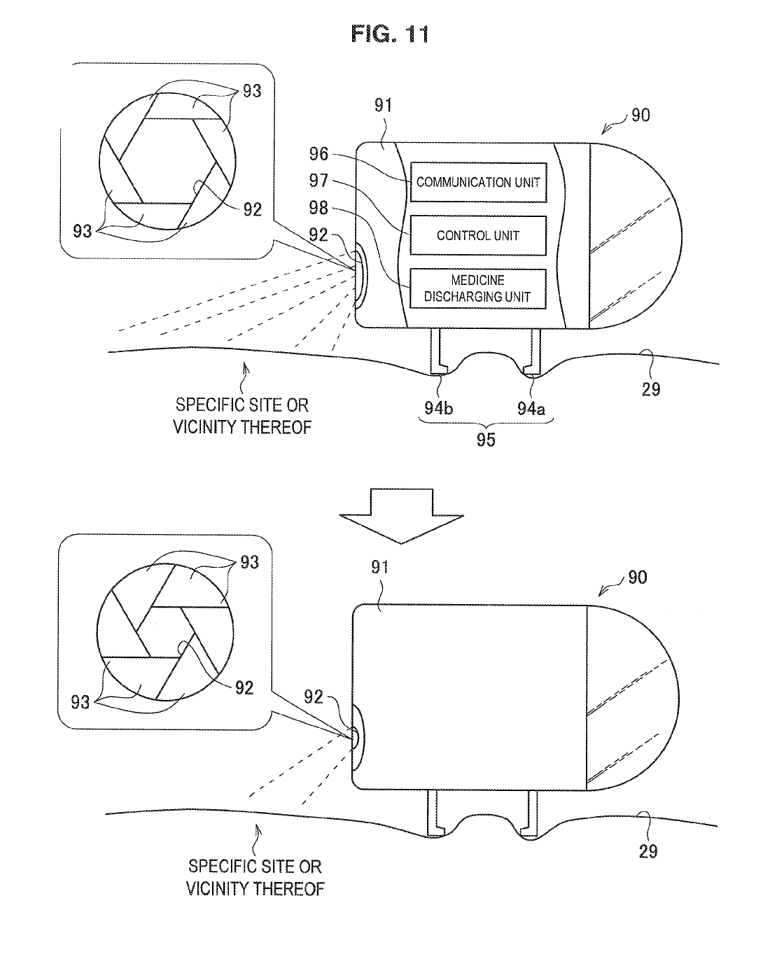

As a modification example 2 of the first embodiment, a capsule type medical device which controls the size of the medicine discharging outlet and keeps discharging the medicine repeatedly will be described below with reference to FIG. 11.

FIG. 11 shows a capsule type medical device according to the modification example 2. As shown in the upper part of FIG. 11, a capsule type medical device 90 (hereinafter referred to as capsule 90) includes a communication unit 96, a control unit 97, a medicine discharging unit 98, and a suspension means 95.

The capsule 90 transmits position information to the control device 2 by use of the communication unit 96 while moving in the intracelom pipeline 29.

Further, when a notification that the capsule 90 has reached the specific site or the vicinity of the specific site is received from the control device 2, the control unit 97 causes the suspension means 95 to operate, and arms 94a and 95b pinch a mucous membrane inside the intracelom pipeline 29, thereby being suspended at the specific site or in the vicinity of the specific site. Note that a structure of the suspension means 95 is the same as the structure of the suspension means 57 described above for the capsule 1 of an arm type shown in FIG. 5.

Next, when a medicine discharging signal is received from the control device 2, the medicine discharging unit 98 discharges a stored medicine to the specific site or the vicinity of the specific site through an opening 92 provided on an outer case 91. Here, the opening 92 according to this modification example is formed by a plurality of blade members 93 as shown in the upper part of FIG. 11.

Accordingly, the control unit 97 of the capsule 90 can perform control such that the opening 92 is narrowed by the plurality of blade members 93, from the state of the opening 92 shown in the upper part of FIG. 11 to the state of the opening 92 shown in the lower part of FIG. 11, thereby narrowing a medicine discharged range gradually.

Accordingly, the capsule 90 can discharge the medicine to a wide range when the medicine discharge starts, and can narrow the medicine discharged range with the passage of time so as to discharge the medicine to a pin point.

In this manner, the capsule 90 according to the present disclosure can control the discharged amount of medicine or the medicine discharged range so that the medicine can be discharged repeatedly (sustainably) to the specific site or the vicinity of the specific site. Note that in the example shown in FIG. 11, the size of the opening 92 is controlled by the plurality of blade members 93; however, this is just an example, and there is no particular limitation on a structure in which the size of the opening 92 is controlled.

The first embodiment has been described above in detail. According to the above described first embodiment, the capsules 1, 60, and 90 can perform treatment of discharging the medicine continuously while being suspended at the specific site or in the vicinity of the specific site.

Note that as methods of discharging the medicine, not only a method of spraying the medicine to the specific site or the vicinity of the specific site, as in the capsules 1, 60, and 90, but also a method of applying the medicine directly onto the specific site or the vicinity of the specific site, for example, may be employed.

Further, the position of each of the capsules 1, 60, and 90 described above is detected by the control device 2, it is determined whether or not each of the capsules 1, 60, and 90 has reached the specific site or the vicinity of the specific site, and each of the capsules 1, 60, and 90 discharges the medicine continuously under control of the control device 2 (in accordance with the generation of the external rotating magnetic field and the transmission of the discharge signal). However, the medical system according to the present disclosure is not limited to such a center management type, and may be an autonomous type in which the capsule is autonomous.

In a case of an autonomous capsule, the capsule itself memorizes the registered specific site and detects the position of the capsule while moving in the body cavity. The position of the capsule may be detected through analysis of a photographed image obtained by photographing the inside of the body cavity, for example. Further, the autonomous capsule determines whether or not the capsule has reached the specific site or the vicinity of the specific site, and in a case where it is determined that the capsule has reached the specific site or the vicinity of the specific site, the autonomous capsule causes a suspension means to operate and performs treatment of discharging a medicine continuously while being suspended at the specific site or in the vicinity of the specific site.

In a case of such an autonomous capsule, even in a case where it is necessary to swallow a medicine twice a day, such as in the morning and at night, for example, the autonomous capsule according to the present disclosure is only needed to be swallowed once in the morning. That is, the autonomous capsule according to the present disclosure is introduced into the body cavity of the test object in the morning, is suspended at a portion of the stomach to spray the medicine A, and then, after a predetermined time passes, the capsule can spray the medicine B at night. Accordingly, the test object can receive effective medical treatment by swallowing only one capsule a day.

### 2-2. Second Embodiment

The above first embodiment has shown the treatment of discharging the medicine continuously as an example of continuous treatment; however, continuous treatment according to the present disclosure is not limited thereto, and for example, treatment of giving stimulation continuously may be performed. Here, the stimulation may be heating, cooling, electricity, magnetism, vibration, oxygen discharge, needle stimulation, and the like, for example. In this embodiment, continuous stimulation using (any of) these examples enables more effective medical treatment.

For example, in a case where a survival rate of a specific cell decreases at a specific temperature, local hyperthermia is effective. More specifically, for example, since the survival rate of a cancer cell decreases at 42.5° C., it is effective to perform local hyperthermia for a certain time (about 300 minutes to 500 minutes).

Further, continuous stimulation of the specific site can activate an immune mechanism (can label a killer T cell or activate an NK cell, for example), and lymphocytes and the like can be concentrated to the specific site or the vicinity of the specific site.

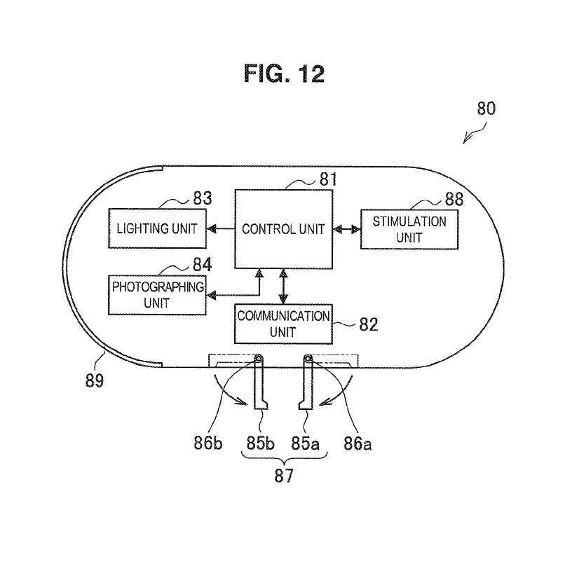

Accordingly, in the second embodiment, a capsule type medical device (e.g., a hyperthermia device) which performs treatment of giving continuous stimulation while being suspended at the specific site is proposed. Thus, more effective medical treatment becomes possible and methods of medical treatment in the medical field can be dramatically improved. A capsule type medical device according to the second embodiment will be described below in detail with reference to FIG. 12.

FIG. 12 shows a configuration of the capsule type medical device according to the second embodiment. As shown in FIG. 12, a capsule type medical device 80 (hereinafter also referred to as capsule 80) includes a control unit 81, a communication unit 82, a lighting unit 83, a photographing unit 84, a suspension means 87, and a stimulation unit 88. The lighting unit 83 and the photographing unit 84 are disposed inside a transparent cover 89, and photographs the inside of the intracelom pipeline while the capsule 80 moves in the body.

The control unit 81 controls each structural element of the capsule 80. For example, in a case where the capsule 80 is a center management type, the control unit 81 causes the communication unit 82 to transmit a signal for position detection to the control device 2. Further, when a notification that the capsule 80 is at the specific site or in the vicinity of the specific site is received from the control device 2, the control unit 81 causes the suspension means 87 to operate, so that the capsule 80 is suspended at the specific site or the vicinity of the specific site. Further, the control unit 81 performs control such that the stimulation unit 88 stimulates the specific site or the vicinity of the specific site in accordance with a control signal received from the control device 2.

Alternatively, in a case where the capsule 80 is an autonomous type, the capsule 81 may analyze a photographed image of the inside of the body, the photographed image being outputted from the photographing unit 84, and may determine whether or not the capsule 80 has reached the specific site or the vicinity of the specific site that is registered in advance. In a case where it is determined that the capsule 80 has reached the specific site or the vicinity of the specific site, the control unit 81 causes the suspension means 87 to operate, so that the capsule 80 is suspended at the specific site or in the vicinity of the specific site. Further, the control unit 81 performs control such that the stimulation unit 88 gives stimulation while being suspended at the specific site or in the vicinity of the specific site.

The suspension means 87 include arms 85a and 85b and pins 86a and 86b. Specifically, the suspension means 87 operates in the same manner as the suspension means 57 which is described above with reference to FIG. 5; the arms 85a and 85b rotate with a fulcrum of the pins 86a and 86b, and pinch a mucous membrane of the inner wall of the intracelom pipeline. Thus, the capsule 80 can be suspended at the specific site or in the vicinity of the specific site. Note that the suspension means 87 shown in FIG. 12 is an example of the suspension means included in the capsule 80, and the suspension means included in the capsule 80 according to the present embodiment is not limited thereto. For example, the capsule 80 may include a balloon type suspension means.

The stimulation unit 88 can stimulate the specific site continuously. More specifically, the stimulation unit 88 may be achieved by a vibration unit which generates minute vibration, a heat generating unit which warms the specific site, a cooling unit which cools the specific site, or the like, for example. Further, the stimulation unit 88 may give stimulation repeatedly (successively for a certain time), or may give stimulation periodically or non-periodically. Further, the stimulation unit 88 may give stimulation at a specified time. Furthermore, the stimulation unit 88 according to the present embodiment may perform control such that a stimulation range is narrowed in accordance with the passage of time, or may perform control such that stimulation intensity is decreased in accordance with the passage of time.

Such a configuration enables the capsule 80 according to the second embodiment to be suspended at the specific site or in the vicinity of the specific site and to give stimulation continuously.

### 3. CONCLUSION

As described above, the medical system according to the present embodiment can perform more effective treatment by performing continuous treatment while being suspended at a predetermined site. Further, this can dramatically improve methods of medical treatment in the medical field.

More specifically, the capsule type medical device according to the first embodiment may discharge a plurality of medicines continuously to the specific site or the vicinity of the specific site, for example.

Further, the capsule type medical device according to the second embodiment may stimulate the specific site or the vicinity of the specific site continuously, thereby killing a specific cell, activating an immune mechanism, or promoting concentration of lymphocytes and the like.

The preferred embodiments of the present invention have been described above with reference to the accompanying drawings, whilst the present invention is not limited to the above examples, of course. A person skilled in the art may find various alterations and modifications within the scope of the appended claims, and it should be understood that they will naturally come under the technical scope of the present invention.

For example, the suspension means included in the capsule according to each embodiment of the present disclosure is not limited to the above described balloon type and arm type. For example, a clip type described in the above Patent Literature 1 (JP 2007-014634A) may be used.

In a case of a clip type, a short string member is connected to the capsule 1, and a clip (a locking unit) is attached to the tip of the string member. Further, at the specific site or in the vicinity of the specific site, the clip of the capsule 1 is directly fixed and secured to a tissue in the body cavity by use of an endoscopic clipping treatment tool. Note that in a case where two clips are attached to the capsule 1 via the respective string members, after one of the clips is fixed to the inside of the body cavity by use of a clipping treatment tool, the other fixing position is adjusted, thereby enabling adjustment of the disposed posture of the capsule 1.

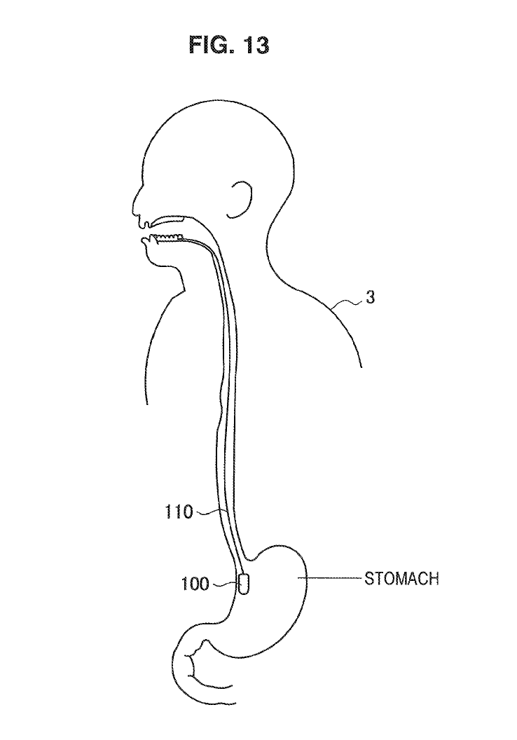

As another suspension means included in the capsule type medical device according to the present disclosure, a linear unit having a predetermined length may be used. For example, as shown in FIG. 13, a linear unit 110 is connected to a capsule type medical device 100 (hereinafter referred to as capsule 100), and the tip of the linear unit 110 is fixed to a mouth portion of the test object 3. Since the linear unit 110 has the length from the mouth portion to the specific site or the vicinity of the specific site, the capsule 100 can be suspended at the specific site or in the vicinity of the specific site.

In a case where the tip of the linear unit 110 is connected to an external power device, the capsule 100 can be supplied with power via the linear unit 110 from the power device.

Furthermore, the capsule type medical device according to the present disclosure may be suspended in accordance with magnetism generated outside. For example, the control device 2 causes an external magnetism generating device to operate, thereby generating magnetism at the specific site or in the vicinity of the specific site. In this case, the capsule type medical device moving in the body cavity of the test object 3 can be suspended at the specific site or in the vicinity of the specific site by the magnetism generated outside.

In each of the embodiments above, as an example of continuous treatment, treatment of discharging a medicine and of giving stimulation has been described; however, continuous treatment according to the present disclosure is not limited thereto. For example, the capsule type medical device according to the present disclosure may observe a healing state while being suspended at the specific site and may perform treatment of discharging a medicine or of giving stimulation in accordance with the healing state, as an example of continuous treatment.

Such a capsule type medical device may observe the healing state through analysis of a photographed image obtained by the photographing unit photographing the specific site or the vicinity of the specific site, or may observe the healing state on the basis of an extracted biological tissue. Alternatively, the capsule type medical device may observe the healing state on the basis of a predetermined value (e.g., a pH level) detected by a sensor that detects the status in the body.

In the above embodiments, as described above with reference to FIG. 7, time intervals are specified as timing of discharging the medicine; however, specification of timing of discharging the medicine according to the present disclosure is not limited thereto. For example, time specification may be “7:00 am, 1:00 pm, 9:00 pm”, for example.

The capsule type medical device according to the present disclosure may discharge the medicine in accordance with mealtime (whether the test object has meal or not). More specifically, whether the test object has meal or not may be notified from the control device 2 or may be determined by the capsule itself. In a case where whether the test object has meal or not is determined by the capsule itself, the determination may be made on the basis of a photographed image of a stomach or the pH level of gastric juices, for example. Thus, it becomes possible to control discharging the medicine before, during, after, and between meals.

Further, the capsule type medical device according to the present disclosure may discharge the medicine in accordance with the sleeping time. The sleeping time may be set in advance. Alternatively, the control device 2 may determine and notify the start and end of sleep of the test object 3 to the capsule, or a medical staff may determine the start and end of sleep of the test object 3 and the control device 2 may notify the start and end of sleep. Thus, it becomes possible to control discharging the medicine before, immediately after, and during the sleep.

Furthermore, the capsule type medical device according to the present disclosure may perform control such that the discharged amount of medicine decreases in accordance with the passage of time.

Furthermore, in the example shown in FIG. 1, the capsule 1 and the control device 2 perform data communication with each other via the extracorporeal unit 7; however, the medical system according to an embodiment of the present disclosure is not limited thereto. For example, the capsule 1 and the control device 2 may perform data communication directly with each other.

Additionally, the present technology may also be configured as below.

(1)

A capsule type medical device which is able to be suspended in a body cavity,

wherein the capsule type medical device performs continuous treatment while being suspended at a predetermined site.

(2)

The capsule type medical device according to (1), including:

a stimulation unit configured to give stimulation continuously to the predetermined site.

(3)

The capsule type medical device according to (2),

wherein the stimulation unit gives stimulation repeatedly.

(4)

The capsule type medical device according to (2),

wherein the stimulation unit gives stimulation periodically or non-periodically.

(5)

The capsule type medical device according to (2),

wherein the stimulation unit gives stimulation at a specified time.

(6)

The capsule type medical device according to any one of (2) to (5),

wherein the stimulation unit performs control in a manner that a stimulation range is narrowed in accordance with passage of time.

(7)

The capsule type medical device according to any one of (2) to (6),

wherein the stimulation unit performs control in a manner that stimulation intensity is decreased in accordance with passage of time.

(8)

The capsule type medical device according to any one of (1) to (7), including:

a medicine discharging unit configured to spray or apply a medicine continuously while being suspended at the predetermined site.

(9)

The capsule type medical device according to (8),

wherein the medicine discharging unit discharges the medicine repeatedly.

(10)

The capsule type medical device according to (8),

wherein the medicine discharging unit discharges the medicine periodically or non-periodically.

(11)

The capsule type medical device according to (8),

wherein the medicine discharging unit discharges the medicine at a specified time.

(12)

The capsule type medical device according to any one of (8) to (11),

wherein the medicine discharging unit performs control in a manner that a discharge range is narrowed in accordance with passage of time.

(13)

The capsule type medical device according to any one of (8) to (12),

wherein the medicine discharging unit performs control in a manner that a discharged amount is decreased in accordance with passage of time.

(14)

The capsule type medical device according to (8),

wherein the medicine discharging unit discharges the medicine in accordance with meal or sleeping time.

(15)

The capsule type medical device according to (8),

wherein the medicine discharging unit performs control in a manner that the medicine is discharged in accordance with a healing state of the predetermined site.

(16)

The capsule type medical device according to any one of (1) to (15),

wherein the capsule type medical device includes
a reception unit configured to receive a control signal from a control device, and

wherein the capsule type medical device performs continuous treatment on the predetermined site in accordance with the control signal received by the reception unit.

(17)

The capsule type medical device according to any one of (1) to (16),

wherein the capsule type medical device includes
a linear unit for being suspended at the predetermined site.
   (18)

The capsule type medical device according to (17),

wherein the capsule type medical device is supplied with power from an external power device via the linear unit.

(19)

The capsule type medical device according to any one of (1) to (18),

wherein the capsule type medical device is suspended at the predetermined site by magnetism.

(20)

A medical system including:

a control device; and

a capsule type medical device configured to be able to be suspended in a body cavity,

wherein the capsule type medical device includes
a reception unit configured to receive a control signal from the control device, and
a treatment unit configured to perform continuous treatment while being suspended at a predetermined site in accordance with the control signal received by the reception unit.

### REFERENCE SIGNS LIST

1, 60, 80, 90, 100 capsule type medical device
2 control device
3 test object
6 rotating magnetic field generating device
7 extracorporeal unit
12 driver circuit
14 electromagnet
21 control unit
22 communication unit
23 display unit
24 operation input unit
25 position detecting unit
26 determination unit
29 intracelom pipeline
30 outer case
31 opening
32 transparent cover
34 photographing optical system
36 photographing sensor
37 control unit
38 memory and communication unit
39 battery
40, 62, 90 storage unit
42 opening
44 first depressed portion
45 screw hole (female screw)
46 second depressed portion
47 moving body storing unit
48 screw portion (male screw portion)
49 cylindrical portion
50 T-shape hole
51 stopper
52 moving body
54 medicine discharging unit
55a, 55b, 75a, 75b, 85a, 85b, 94a, 94b arm
56a, 56b, 76a, 76b, 86a, 86b pin
57, 77, 87, 95 suspension means
61 housing
61a wall portion
64 medicine discharging outlet
66 medicine discharging pipeline
68 switch valve
70 control unit (medicine discharging unit)
71 reception unit
72 transmission unit
73 battery
81, 97 control unit
82, 96 communication unit
83 lighting unit
84 photographing unit
88 stimulation unit
98 medicine discharging unit
92 opening
110 linear unit

1. A capsule type medical device which is able to be suspended in a body cavity,
wherein the capsule type medical device performs continuous treatment while being suspended at a predetermined site.

2. The capsule type medical device according to claim 1, comprising:
a stimulation unit configured to give stimulation continuously to the predetermined site.

3. The capsule type medical device according to claim 2,
wherein the stimulation unit gives stimulation repeatedly.

4. The capsule type medical device according to claim 2,
wherein the stimulation unit gives stimulation periodically or non-periodically.

5. The capsule type medical device according to claim 2,
wherein the stimulation unit gives stimulation at a specified time.

6. The capsule type medical device according to claim 2,
wherein the stimulation unit performs control in a manner that a stimulation range is narrowed in accordance with passage of time.

7. The capsule type medical device according to claim 2,
wherein the stimulation unit performs control in a manner that stimulation intensity is decreased in accordance with passage of time.

8. The capsule type medical device according to claim 1, comprising:
a medicine discharging unit configured to spray or apply a medicine continuously while being suspended at the predetermined site.

9. The capsule type medical device according to claim 8,
wherein the medicine discharging unit discharges the medicine repeatedly.

10. The capsule type medical device according to claim 8,
wherein the medicine discharging unit discharges the medicine periodically or non-periodically.

11. The capsule type medical device according to claim 8,
wherein the medicine discharging unit discharges the medicine at a specified time.

12. The capsule type medical device according to claim 8,
wherein the medicine discharging unit performs control in a manner that a discharge range is narrowed in accordance with passage of time.

13. The capsule type medical device according to claim 8,
wherein the medicine discharging unit performs control in a manner that a discharged amount is decreased in accordance with passage of time.

14. The capsule type medical device according to claim 8,
wherein the medicine discharging unit discharges the medicine in accordance with meal or sleeping time.

15. The capsule type medical device according to claim 8,
wherein the medicine discharging unit performs control in a manner that the medicine is discharged in accordance with a healing state of the predetermined site.

16. The capsule type medical device according to claim 1,
wherein the capsule type medical device includes
a reception unit configured to receive a control signal from a control device, and
wherein the capsule type medical device performs continuous treatment on the predetermined site in accordance with the control signal received by the reception unit.

17. The capsule type medical device according to claim 1,
wherein the capsule type medical device includes
a linear unit for being suspended at the predetermined site.

18. The capsule type medical device according to claim 17,
wherein the capsule type medical device is supplied with power from an external power device via the linear unit.

19. The capsule type medical device according to claim 1,
wherein the capsule type medical device is suspended at the predetermined site by magnetism.

20. A medical system comprising:
a control device; and
a capsule type medical device configured to be able to be suspended in a body cavity,
wherein the capsule type medical device includes
a reception unit configured to receive a control signal from the control device, and
a treatment unit configured to perform continuous treatment while being suspended at a predetermined site in accordance with the control signal received by the reception unit.
